(19) United States
(12) Patent Application Publication
(10) Pub. No.: US 2026/0241384 A1
(43) Pub. Date: Aug. 20, 2026

(54) MICROWELL ARRAY AND METHODS OF USE

(71) Applicant: THE RESEARCH FOUNDATION FOR THE STATE UNIVERSITY OF NEW YORK, Albany, NY (US)

(72) Inventor: Eric BROUZES, Port Jefferson, NY (US)

(73) Assignee: THE RESEARCH FOUNDATION FOR THE STATE UNIVERSITY OF NEW YORK, Albany, NY (US)

(21) Appl. No.: 19/471,114

(22) PCT Filed: Mar. 29, 2024
(86) PCT No.: PCT/US2024/022119

§ 371 Date: Sep. 30, 2025

Related U.S. Application Data
(60) Provisional application No. 63/456,163, filed on Mar. 31, 2023.

(51) Int. Cl. B01L 3/00; C12N 15/10

(52) U.S. Cl. CPC B01L 3/5085; C12N 15/1065; B01L 2200/0647; B01L 2200/0689; B01L 2300/0681; B01L 2300/0819

(57) ABSTRACT

The present disclosure is directed to a microwell array comprising a plurality of wells allowing for single cell genomics and methods for using such a microwell array in single cell genomics.

### BACKGROUND OF THE DISCLOSURE

Single-cell genomics enables the study of biological functions at the individual cell level in an unbiased and highly multiplex manner. It contrasts with conventional single-cell methods such as flow cytometry, microscopy, or fluorescent in-situ hybridization. Single-cell genomics has enabled us to examine the role of heterogeneity in many different tissues to address fundamental biological questions in diverse fields such as cancer, neurosciences, and developmental biology. Recently, single-cell genomics has expanded further into epigenomics, proteomics, and multi-omics. Such breakthroughs offer the potential to expand the understanding of collections of genes and proteins with relevance to basic and disease biology.

Single-cell genomic methods essentially isolate single cells and barcode their genetic material before sequencing. High-throughput methods use microfluidic droplets (Drop-seq) and arrays of microwells (seq-Well) for single-cell isolation. Barcoding is performed by way of barcoded beads that carry the unique DNA tag to be appended to the cell genetic information. Poisson's statistics limit barcoded bead-based methods, and more than 90% of costly beads are wasted. Recent alternatives use single-cells transformed into hydrogel beads (Bag-seq) or fixed single-cells (Split-seq) as the substrates for barcoding by split-pooling. Hydrogel beads containing nucleic acids are known as “BAGs” (see S. Li, J. Kendall, S. Park, Z. Wang, J. Alexander, A. Moffitt, N. Ranade, C. Danyko, B. Gegenhuber, S. Fischer, B. D. Robinson, H. Lepor, J. Tollkuhn, J. Gillis, E. Brouzes, A. Krasnitz, D. Levy, M. Wigler, Genome Res, 2020,30, 49-61.) While these methods address the limitations of cell-bead coupling, they require intensive hands-on labor.

Despite its benefit, single-cell genomics is hindered by a laborious manual and low throughput workflow. In addition, the required number of cells to analyze can also be difficult to attain with existing methods. One of the key drivers of the growth of the single-cell genomics market is the development of innovative platforms to streamline the sample preparation of single-cells at high throughput.

Current methods of single cell genomics are based on three principles. First, arrays of on-chip valves, limiting the number of cells that can be processed (usually a maximum of 96 cells); 2-microfluidic droplets relying on complicated actuation and manipulations, and are unable to simply address applications such as single-cell quantitative (q) PCR or Reverse Transcription (RT)-PCR; or 3-robotic based system that do not solve the throughput and simplicity issues. On a technical level, the main limitations of those methods are the limited number of cells analyzed and the limited number of times the systems can perform washes. As a result, workflows that can be used on those platforms are limited to very few steps (1 or 2), excluding applications such as single cell epigenomics.

The disclosed methods and devices are configured to undertake both cell isolation and sample preparation. The disclosed apparatus will allow multi-step processing of massive numbers of single cells for single-cell genomics (100,000's). The present disclosure will also enable single-cell qRT-PCR and digital PCR.

### SUMMARY OF THE DISCLOSURE

The present disclosure is directed to a microwell array allowing for single cell genomics and methods for using such a microwell array in single cell genomics.

In a first aspect, the present disclosure is directed to a microwell array comprising a first surface, a second surface, and a connection area, where the first surface and second surface meet at the connection area; each surface of the microwell array comprises a plurality of independent wells wherein: each well comprises a top, bottom, and sides; each well of the first surface is connected to a corresponding well of the second surface, wherein the wells are configured so that the bottom of the well of the first surface is in contact with the bottom of the well of the second surface and the contact takes place at the connection area; and wherein the microarray enables coupling of one particle with at least one bead. In one embodiment, the microwell array comprises microwells that are circular, triangular, rectangular, pentangular, hexangular, heptangular, or octangular. In some embodiments, the microwell array comprises microwells where the top of the each microwell is open. In some embodiments, the microwell array comprises microwells where the bottom of each microwell is open. In some embodiments, microwell array comprises a porous membrane between the first surface and second surface in the connection area. In some embodiments, the microwell array comprises microwells where the sides of each well are non-porous. In some embodiments, the microwell array comprises microwells where the sides of each microwell are hydrophilic. In some embodiments, the microwell array comprises microwells with a diameter of at least 1.25-fold the size of the particle placed in the well. In some embodiments, the microwells have a diameter of at least 1.25 to 5-fold, at least 1.25 to 2-fold, at least 1.25 to 3-fold, or at least 1.25 to 4-fold the size of a particle to be placed in the well. In some embodiments, the microwells of the array have a diameter of at least 5 μm. In some embodiments, the microwells of the first surface of the array have a different diameter from the microwells of the second surface of the array. In some embodiments, the microwells of the first surface of the array and the microwells of the second surface of the array have the same diameter. In some embodiments, the microwells of the array have a depth that is smaller than the size of a particle to be placed in the well. In some embodiments, the microwells of the array have a depth that is larger than the size of a particle to be placed in the well. In some embodiments, the microwells of the array have a depth of at least 0.9-fold the size of the particle to be placed in the well. In some embodiments, the microwells of the array have a depth within 0.9 to 5-fold the size of a particle to be placed in the well. In some embodiments, the microwells of the array have a depth of about 1.5-fold the size of a particle to be placed in the well. In some embodiments, the microwells of the first surface of the array and the microwells of the second surface of the array have different depths. In some embodiments, the microwells of the first surface of the array and microwells of the second surface of the array have the same depth. In some embodiments, the bottom of the microwells of the first surface of the array have a diameter of at least 0.2 microns. In some embodiments, the bottom of the microwells of the first surface of the array have a diameter in the range of 0.2 microns to 20 microns. In some embodiments, the bottom of the microwells of the first surface of the array have a diameter in the range of 0.2 microns to 10 microns. In some embodiments, the bottom of the microwells of the first surface of the array have a diameter in the range of 0.2 microns to 12 microns. In some embodiments, the bottom of the microwells of the first surface of the array have a diameter in the range of 0.2 microns to 5 microns. In some embodiments, the bottom of the microwells of the second surface of the array have a diameter of at least 0.2 microns. In some embodiments, the bottom of the microwells of the second surface of the array have a diameter in the range of 0.2 microns to 20 microns. In some embodiments, the bottom of the microwells of the second surface of the array have a diameter in the range of 0.2 microns to 10 microns. In some embodiments, the bottom of the microwells of the second surface of the array have a diameter in the range of 0.2 microns to 12 microns. In some embodiments, the bottom of the microwells of the second surface of the array have a diameter in the range of 0.2 microns to 5 microns. In some embodiments, the porous membrane in the connection area comprises pores ranging in size from 0.2 microns to 20 microns. In some embodiments, the porous membrane in the connection area comprises pores ranging in size from 0.2 microns to 10 microns. In some embodiments, the porous membrane in the connection area comprises pores ranging in size from 0.2 microns to 12 microns. In some embodiments, the porous membrane in the connection area comprises pores ranging in size from 0.2 microns to 5 microns. In some embodiments, the microwells of one surface of the array comprise particles. In some embodiments, particles can be genomic barcodes. In some embodiments, the particles can be cells (e.g. single cells), tissue particles or beads and in some embodiments the particles can be “BAGs” (hydrogel beads which comprise all the nucleic acids of a cell). The particles can be located on beads in the microwells of one surface. In some embodiments, the barcodes are located in the microwells of the first surface of the array. In some embodiments, the array comprises a sealing device for the first surface of the array. In some embodiments, the sealing device for the first surface is temporary, removable, replaceable, and/or discardable. In some embodiments, the sealing device for the first surface is selected from glass, oil, adhesive, elastomer cover, and porous membrane. In some embodiments, the microwell array comprises a sealing device for the second surface of the array. In some embodiments, the sealing device for the second surface is temporary, removable, replaceable, and/or discardable. In some embodiments, the sealing device for the second surface is selected from glass, oil, adhesive, elastomer cover, and porous membrane.

In another aspect, the present disclosure is directed to a method of barcoding the genetic material of particles, the method comprising three steps: 1) loading one particle to one of a plurality of wells of a microwell array where the microwell array comprises a first surface, a second surface, and a connection area, where the first surface and second surface meet at the connection area; each surface of the microwell array comprises a plurality of independent wells wherein: each well comprises a top, bottom, and sides; each well of the first surface is connected to a corresponding well of the second surface, wherein the wells are configured so that the bottom of the well of the first surface is in contact with the bottom of the well of the second surface and the contact takes place at the connection area; and wherein the microarray enables a the coupling of one particle with at least one bead; 2) processing the barcode to particle coupling; 3) collecting barcoded genetic material. In one embodiment, the method further comprises the steps of: a) loading particles in the wells of the first surface of the microwell array; b) sealing the first surface of the microwell array; c) flipping over the microwell array so that the second surface is accessible; wherein steps a, b, and c occur in succession before step 1. In some embodiments, step a) further comprises loading the particles genomic barcodes into the wells of the first surface of the microwell array by loading a singular capture bead from a plurality of capture beads into a well of the microwell array, wherein the capture beads comprise the particles. In some embodiments, the plurality of capture beads is selected from hydrogel, alginate, silica, glass, magnetic, polystyrene, polyactide, and polyacrylamide. In some embodiments, step a) is performed with a vacuum applied to the microarray. In some embodiments step a) is gravity-driven. In some embodiments, the method further comprises removal of excess loaded particles; wherein the removal of excess loaded particles occurs after step a) but before step b). In some embodiments, the processing step comprises: lysing cells; capturing mRNA molecules through a heating and cooling cycle; releasing the genetic barcodes; hybridizing the genomic material of the cell to the genetic barcode. In some embodiments, the processing step comprises: lysing cells; capturing mRNA molecules through a heating and cooling cycle; hybridizing the genomic material of the cell to the genetic barcode; and releasing the genetic barcodes. In some embodiments, the method further comprises exposing the microarray to a form of centrifugation.

In some embodiments, the method further comprises 1a) sealing the loaded cells in the wells of the second surface of the microwell array; wherein step 1a) occurs after step 1) and before step 2). In some embodiments, the loaded cells can be sealed with a porous or non-porous membrane. In some embodiments, the method further comprises a step of adding one or more buffers and/or reagents to at least one well; where the at least one well comprises a particle and at least one genetic barcode; and wherein the step of adding one or more buffers and/or reagents to the at least one well occurs after step 1) and before step 2). In some embodiments, the method further comprises a step of step of adding one or more buffers and/or reagents to at least one well; where the at least one well comprises a particle and at least one genetic barcode; and wherein the step of adding one or more buffers and/or reagents to the at least one well occurs step 1a). In some embodiments, the one or more buffers and/or reagents comprise a lysing agent. In some embodiments, the method further comprises opening the array and washing the barcoded genomic material. In some embodiments, the method further comprises, a step of removing excess cells from the second surface of the microarray, wherein the step of removing excess cells occurs after step 1) and before step 2). In some embodiments, the method further comprises performing reverse-transcription (RT) with reverse template switch. In some embodiments, the method further comprises performing polymerase chain reaction directly on the array or after collecting barcoded genomic material. In some embodiments, the method further comprises sequencing of captured genomic material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure will be better understood by reference to the following drawings, which are provided as illustrative of certain embodiments of the subject application, and not meant to limit the scope of the present disclosure.

FIG. 1A-B. FIG. 1A shows a cross-section of a microwell comprising side wings. FIG. 1B shows a schematic of a top view of a microwell comprising side wings.

FIG. 2A-E. Data demonstrate the validity of the microwell array design. A) Schematic cross-section of a microwell loaded with beads of different sizes. B-C) Field of view focused on small beads. D-E) Same field of view focused on large beads. C, E) Composite of the brightfield and the red fluorescence images. In the field of view, four perfect cell-bead pairings are seen, one microwell that contains only a small bead (top right of the field of view) and one empty microwell (bottom left of the field of view). The scale bar represents 50 μm.

FIG. 3 shows schematic of one embodiment of the method where: 1) barcoded capture beads are loaded; 2) the first surface of the array is sealed; 3) the array is flipped; 4) cells are loaded; 5) the second surface of the array is sealed; 6) cells are lysed and mRNA captured by a heating (50° C.) and a slow cooling cycle; 7) the second surface of the array is opened and washed; 8) reverse-transcription (RT) with reverse template switch is performed; 9) PCR is performed directly on the array or after collecting beads.

FIG. 4A-E shows a schematic of one embodiment of the flip array method where a) vacuum or flow-driven particles, i.e. genomic barcodes are loaded in the wells of the first surface of the microwell array; b) the first surface of the microwell array is sealed with a porous or non-porous membrane; c) the microwell array is flipped over so that the second surface is accessible; d) particles are loaded in empty microwells using gravity only with non-porous membranes or using gravity or vacuum-driven loading with a porous membrane; e) and the microwells are optionally sealed to proceed with cell lysis, molecule capture by the beads and unsealed to perform wash and buffer exchange steps to lead to biomolecular and biochemical reactions.

FIG. 5 shows a schematic of active cell loading with a vacuum (left) versus passive cell loading with gravity (right).

FIG. 6A-F.) First surface (bead side) and second surface (cell side) of one embodiment of the microwell array. The capture rates from both sides are different because of the discrepancy in microwell densities. B) Design for the bead/cell capture rate study. C) Microwell array designed to increase and match the densities of barcode bead and cell microwells. The hexagonal microwells include a narrow trench at their bottom (green and red). Those trenches are rotated by 90° for the bottom layer (red). D) This design allows the creation of a conduit between a single cell microwell with a single bead microwell even if the arrays are grossly misaligned. E) Schematic of the microfabrication process in which the UV epoxy is sandwiched between two PDMS molds before curing. F) Schematic of microfabrication process by molding of a UV-curable material using a soft (PDMS) die.

### DETAILED DESCRIPTION OF THE DISCLOSURE

In the discussion and claims herein, the term “about” indicates that the value listed may be somewhat altered, as long as the alteration does not result in nonconformance of the process or device. For example, for some elements the term “about” can refer to a variation of ±0.1%, for other elements, the term “about” can refer to a variation of ±1% or ±10%, or any point therein.

As used herein, the term “substantially”, or “substantial”, is equally applicable when used in a negative connotation to refer to the complete or near complete lack of an action, characteristic, property, state, structure, item, or result. For example, a surface that is “substantially” flat would either completely flat, or so nearly flat that the effect would be the same as if it were completely flat.

As used herein terms such as “a”, “an” and “the” are not intended to refer to only a singular entity, but include the general class of which a specific example may be used for illustration.

As used herein, terms defined in the singular are intended to include those terms defined in the plural. Additionally, terms defined in the plural are intended to include those terms defined in the singular.

References in the specification to “one embodiment”, “certain embodiments”, some embodiments” or “an embodiment”, indicate that the embodiment(s) described may include a particular feature or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. For purposes of the description hereinafter, the terms “upper”, “lower”, “right”, “left”, “vertical”, “horizontal”, “top”, “bottom”, and derivatives thereof shall relate to the invention, as it is oriented in the drawing figures. The terms “overlying”, “atop”, “positioned on” or “positioned atop” means that a first element, is present on a second element, wherein intervening elements interface between the first element and the second element. The term “direct contact” or “attached to” means that a first element, and a second element, are connected without any intermediary element at the interface of the two elements.

Reference herein to any numerical range expressly includes each numerical value (including fractional numbers and whole numbers) encompassed by that range. To illustrate, reference herein to a range of “at least 50” or “at least about 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” or “less than about 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc.

As used herein, the term “microwell,” generally refers to a well with a volume of less than about 1 mL. Microwells may be made in various volumes, larger or smaller than about 1 mL, depending on the application. In some embodiments, the term “microwell” may refer to a topological feature such as a well, a pit, a depression and similar, in which at least one of the linear dimensions is greater than about 1 micron but smaller than about 1 mm. However, in some embodiments, wells with linear dimensions greater than 1 mm may be also referred to as microwells.

As used herein “hydrophilic” refers to molecules and/or components of molecules having at least one hydrophilic group. As used herein, “hydrophobic” refers to molecules and/or components of molecules having at least one hydrophobic group. Hydrophilic molecules or components thereof tend to have ionic and/or polar groups, and hydrophobic molecules or components thereof tend to have nonionic and/or nonpolar groups. Hydrophilic molecules or components thereof tend to participate in stabilizing interactions with an aqueous solution, including hydrogen bonding and dipole-dipole interactions. Hydrophobic molecules or components tend not to participate in stabilizing interactions with an aqueous solution and, thus often cluster together in an aqueous solution to achieve a more stable thermodynamic state.

As used herein “fluorophilic” refers to molecules and/or components of molecules having at least one fluorophilic group. A fluorophilic group is one that is capable of participating in stabilizing interactions with a fluorous phase. Fluorophilic groups useful in block copolymers of the present disclosure include, but are not limited to, fluorocarbon groups, perfluorinated groups and semifluorinated groups.

As used herein, “Poisson's statistics” or “Poisson's distribution” defines a discrete probability distribution that expresses the probability of a given number of events occurring in a fixed interval of time or space if these events occur with a known constant mean rate and independently of the time since the last event. A discrete random variable X is said to have a Poisson distribution, with parameter >0, if it has a probability mass function given by:

$$f ( k ; λ = P r ( X = k ) = \frac{{λ}^{k} {e}^{- λ}}{k !}$$

where
k is the number of occurrences (k=0,1,2, . . . )
e is Euler's number (e=2.71828 . . . )
! is the factorial function.

The positive real number λ is equal to the expected value of X and also to its variance.

$$λ = E ( X ) = Var ( X )$$

The present disclosure includes devices and methods for cell capture and/or analysis. These devices have numerous applications in such diverse fields as developmental biology, immunology, neuroscience, cancer research, basic cell biology, stem cell biology and microbiology.

### Microwell Array

In a first aspect, the present disclosure is directed to a microwell array comprising a first surface, a second surface, and a connection area, where the first surface and second surface meet at the connection area; each surface of the microwell array comprises a plurality of independent wells wherein: each well comprises a top, bottom, and sides; each well of the first surface is connected to a corresponding well of the second surface, wherein the wells are configured so that the bottom of the well of the first surface is in contact with the bottom of the well of the second surface, and the contact between the bottom of the well of the first surface and the bottom of the well of the second surface takes place at the connection area.

In some embodiments, the microarray enables coupling of one particle with at least one barcode. In some embodiments, the barcode is attached to a bead. In some embodiments, the barcode is directly in the microwell. In some embodiments, the microarray enables coupling of one particle with a plurality of barcodes. In some embodiments, the microarray enables coupling of one particle with a plurality of beads.

In some embodiments, the microwell array comprises microwells that are circular, triangular, rectangular, pentangular, hexangular, septangular, or octangular in shape.

In some embodiments, the microwell array comprises microwells where the top of each microwell is open.

In some embodiments, the microwell array comprises microwells where the bottom of each microwell is open.

In some embodiments, the microwell array comprises a membrane between the first surface and second surface in the connection area. In some embodiments, the membrane is porous. In some embodiments the microwell array comprises a porous membrane and a non-porous membrane. The porous membrane can be positioned on the top (e.g. first or second surface) or on the bottom (e.g. first or second surface) of the microwell. In some embodiments the microwell array comprises a porous membrane on the top surface and the bottom surface of the microwell. In some embodiments the microwell array comprises a non-porous membrane on the top surface and the bottom surface of the microwell. In an alternative embodiment, the non-porous membrane can be positioned on the top or on the bottom of the microwell. In some embodiments the porous membrane can be formed from any thermoplastic material, such as a track etched polycarbonate, polystyrene, polymethacrylate, cyclic olefin copolymer, cyclic olefin polymers. In some embodiments, a non-porous membrane comprises glass, oil, adhesive, elastomer cover and/or PCR tape.

In some embodiments, the microwells of the microwell array comprise a top, bottom, and sides. The top and bottom of each microwell has a diameter. The diameter at the top of the microwell can be the same as the diameter at the bottom of the microwell. In some embodiments, the diameter at the top of the microwell is different than the diameter at the bottom of the microwell.

In some embodiments, the microwell array comprises microwells with a diameter at the top of the microwell of at least 1.5-fold the size of the particle placed in the well. In some embodiments, the diameter of the microwell at the top of the microwell is between 1.5 to 5-fold the size of a particle to be placed in the well. In some embodiments, the diameter of the microwell at the top of the microwell is about 1.5-fold the size of a particle to be placed in the well. In some embodiments, the diameter of the microwell at the top of the microwell is about 1.6-fold the size of a particle to be placed in the well. In some embodiments, the diameter of the microwell at the top of the microwell is about 1.7-fold the size of a particle to be placed in the well. In some embodiments, the diameter of the microwell at the top of the microwell is about 1.8-fold the size of a particle to be placed in the well. In some embodiments, the diameter of the microwell at the top of the microwell is about 1.9-fold the size of a particle to be placed in the well. In some embodiments, the diameter of the microwell at the top of the microwell is about 2-fold the size of a particle to be placed in the well. In some embodiments, the diameter of the microwell at the top of the microwell is about 2.1-fold the size of a particle to be placed in the well. In some embodiments, the diameter of the microwell at the top of the microwell is about 2.2-fold the size of a particle to be placed in the well. In some embodiments, the diameter of the microwell at the top of the microwell is about 2.3-fold the size of a particle to be placed in the well. In some embodiments, the diameter of the microwell at the top of the microwell is about 2.4-fold the size of a particle to be placed in the well. In some embodiments, the diameter of the microwell at the top of the microwell is about 2.5-fold the size of a particle to be placed in the well. In some embodiments, the diameter of the microwell at the top of the microwell is about 2.6-fold the size of a particle to be placed in the well. In some embodiments, the diameter of the microwell at the top of the microwell is about 2.7-fold the size of a particle to be placed in the well. In some embodiments, the diameter of the microwell at the top of the microwell is about 2.8-fold the size of a particle to be placed in the well. In some embodiments, the diameter of the microwell at the top of the microwell is about 2.9-fold the size of a particle to be placed in the well. In some embodiments, the diameter of the microwell at the top of the microwell is about 3.0-fold the size of a particle to be placed in the well. In some embodiments, the diameter of the microwell at the top of the microwell is about 3.1-fold the size of a particle to be placed in the well. In some embodiments, the diameter of the microwell at the top of the microwell is about 3.2-fold the size of a particle to be placed in the well. In some embodiments, the diameter of the microwell at the top of the microwell is about 3.3-fold the size of a particle to be placed in the well. In some embodiments, the diameter of the microwell at the top of the microwell is about 3.4-fold the size of a particle to be placed in the well. In some embodiments, the diameter of the microwell at the top of the microwell is about 3.5-fold the size of a particle to be placed in the well. In some embodiments, the diameter of the microwell at the top of the microwell is about 3.6-fold the size of a particle to be placed in the well. In some embodiments, the diameter of the microwell at the top of the microwell is about 3.7-fold the size of a particle to be placed in the well. In some embodiments, the diameter of the microwell at the top of the microwell is about 3.8-fold the size of a particle to be placed in the well. In some embodiments, the diameter of the microwell at the top of the microwell is about 3.9-fold the size of a particle to be placed in the well. In some embodiments, the diameter of the microwell at the top of the microwell is about 4.0-fold the size of a particle to be placed in the well. In some embodiments, the diameter of the microwell at the top of the microwell is about 4.1-fold the size of a particle to be placed in the well. In some embodiments, the diameter of the microwell at the top of the microwell is about 4.2-fold the size of a particle to be placed in the well. In some embodiments, the diameter of the microwell at the top of the microwell is about 4.3-fold the size of a particle to be placed in the well. In some embodiments, the diameter of the microwell at the top of the microwell is about 4.4-fold the size of a particle to be placed in the well. In some embodiments, the diameter of the microwell at the top of the microwell is about 4.5-fold the size of a particle to be placed in the well. In some embodiments, the diameter of the microwell at the top of the microwell is about 4.6-fold the size of a particle to be placed in the well. In some embodiments, the diameter of the microwell at the top of the microwell is about 4.7-fold the size of a particle to be placed in the well. In some embodiments, the diameter of the microwell at the top of the microwell is about 4.8-fold the size of a particle to be placed in the well. In some embodiments, the diameter of the microwell at the top of the microwell is about 4.9-fold the size of a particle to be placed in the well. In some embodiments, the diameter of the microwell at the top of the microwell is about 5.0-fold the size of a particle to be placed in the well. In some embodiments, the microwells of the array have a diameter at the top of the microwell of at least 15 μm. In some embodiments, the microwells of the first surface of the array and the microwells of the second surface of the array have the same diameters at the top and bottom of the wells. In some embodiments, the microwells of the first surface of the array have a different top diameter from the top diameter of the microwells of the second surface of the array. In some embodiments, the microwells of the first surface of the array have a different bottom diameter from the bottom diameter of the microwells of the second surface of the array. In some embodiments, the microwells of the first surface of the array have a different top diameter and a different bottom diameter from the top and bottom diameter of the microwells of the second surface of the array.

In some embodiments, connection area between the microwells have at least one pore with a diameter of at least 0.2 microns. In some embodiments, the connection area between the microwells have at least one pore with a diameter in the range of 0.2 microns to 10 microns. In some embodiments, the connection area between the microwells have at least one pore with a diameter of at least 0.2 microns. In some embodiments, the connection area between the microwells have at least one pore with a diameter in the range of 0.2 microns to 10 microns. In some embodiments, the connection area between the microwells have the same diameter. In some embodiments, the connection area between the microwells have a different diameter.

In some embodiments, the microwell array comprises microwells having sides. The sides of the microwells connect the top of the microwell to the bottom of the microwell. The depth of a microwell is the measurement between the top of the microwell and the bottom of the microwell. In other words, the depth of the microwell is equal to the measurement of the sides of the microwell.

In some embodiments, the microwells of the array have a depth of at least 0.9-fold the size of the particle to be placed in the well. In some embodiments, the microwells have a depth that is smaller than the size of a particle to be placed in the well. In some embodiments, the microwells have a depth that is larger than the size of a particle to be placed in the well. In some embodiments, the microwells of the array have a depth within the range of 0.9 to 5-fold the size of a particle to be placed in the well. In some embodiments, the microwells of the array have a depth of about 0.9-fold the size of a particle to be placed in the well. In some embodiments, the microwells of the array have a depth of about 1.0-fold the size of a particle to be placed in the well. In some embodiments, the microwells of the array have a depth of about 1.1-fold the size of a particle to be placed in the well. In some embodiments, the microwells of the array have a depth of about 1.2-fold the size of a particle to be placed in the well. In some embodiments, the microwells of the array have a depth of about 1.3-fold the size of a particle to be placed in the well. In some embodiments, the microwells of the array have a depth of about 1.4-fold the size of a particle to be placed in the well. In some embodiments, the microwells of the array have a depth of about 1.5-fold the size of a particle to be placed in the well. In some embodiments, the microwells of the array have a depth of about 1.6-fold the size of a particle to be placed in the well. In some embodiments, the microwells of the array have a depth of about 1.7-fold the size of a particle to be placed in the well. In some embodiments, the microwells of the array have a depth of about 1.8-fold the size of a particle to be placed in the well. In some embodiments, the microwells of the array have a depth of about 1.9-fold the size of a particle to be placed in the well. In some embodiments, the microwells of the array have a depth of about 2-fold the size of a particle to be placed in the well. In some embodiments, the microwells of the array have a depth of about 2.1-fold the size of a particle to be placed in the well. In some embodiments, the microwells of the array have a depth of about 2.2-fold the size of a particle to be placed in the well. In some embodiments, the microwells of the array have a depth of about 2.3-fold the size of a particle to be placed in the well. In some embodiments, the microwells of the array have a depth of about 2.4-fold the size of a particle to be placed in the well. In some embodiments, the microwells of the array have a depth of about 2.5-fold the size of a particle to be placed in the well. In some embodiments, the microwells of the array have a depth of about 2.6-fold the size of a particle to be placed in the well. In some embodiments, the microwells of the array have a depth of about 2.7-fold the size of a particle to be placed in the well. In some embodiments, the microwells of the array have a depth of about 2.8-fold the size of a particle to be placed in the well. In some embodiments, the microwells of the array have a depth of about 2.9-fold the size of a particle to be placed in the well. In some embodiments, the microwells of the array have a depth of about 3.0-fold the size of a particle to be placed in the well. In some embodiments, the microwells of the array have a depth of about 3.1-fold the size of a particle to be placed in the well. In some embodiments, the microwells of the array have a depth of about 3.2-fold the size of a particle to be placed in the well. In some embodiments, the microwells of the array have a depth of about 3.3-fold the size of a particle to be placed in the well. In some embodiments, the microwells of the array have a depth of about 3.4-fold the size of a particle to be placed in the well. In some embodiments, the microwells of the array have a depth of about 3.5-fold the size of a particle to be placed in the well. In some embodiments, the microwells of the array have a depth of about 3.6-fold the size of a particle to be placed in the well. In some embodiments, the microwells of the array have a depth of about 3.7-fold the size of a particle to be placed in the well. In some embodiments, the microwells of the array have a depth of about 3.8-fold the size of a particle to be placed in the well. In some embodiments, the microwells of the array have a depth of about 3.9-fold the size of a particle to be placed in the well. In some embodiments, the microwells of the array have a depth of about 4.0-fold the size of a particle to be placed in the well. In some embodiments, the microwells of the array have a depth of about 4.1-fold the size of a particle to be placed in the well. In some embodiments, the microwells of the array have a depth of about 4.2-fold the size of a particle to be placed in the well. In some embodiments, the microwells of the array have a depth of about 4.3-fold the size of a particle to be placed in the well. In some embodiments, the microwells of the array have a depth of about 4.4-fold the size of a particle to be placed in the well. In some embodiments, the microwells of the array have a depth of about 4.5-fold the size of a particle to be placed in the well. In some embodiments, the microwells of the array have a depth of about 4.6-fold the size of a particle to be placed in the well. In some embodiments, the microwells of the array have a depth of about 4.7-fold the size of a particle to be placed in the well. In some embodiments, the microwells of the array have a depth of about 4.8-fold the size of a particle to be placed in the well. In some embodiments, the microwells of the array have a depth of about 4.9-fold the size of a particle to be placed in the well. In some embodiments, the microwells of the array have a depth of about 5.0-fold the size of a particle to be placed in the well. In some embodiments, the microwells of the first surface of the array and the microwells of the second surface of the array have different depths. In some embodiments, the microwells of the first surface of the array and the microwells of the second surface of the array have the same depth.

In some embodiments, the side wall has a curved surface. In some embodiments, the side wall comprises at least one flat surface. In some embodiments, the microwell array comprises microwells that are circular, triangular, rectangular, pentangular, hexangular, heptangular, or octangular in shape. In some embodiments, the microwell array comprises microwells that are circular in shape. In some embodiments, the microwell array comprises microwells that are triangular in shape. In some embodiments, the microwell array comprises microwells that are rectangular in shape. In some embodiments, the microwell array comprises microwells that are pentangular in shape. In some embodiments, the microwell array comprises microwells that are hexangular in shape. In some embodiments, the microwell array comprises microwells that are heptangular in shape. In some embodiments, the microwell array comprises microwells that are octangular in shape.

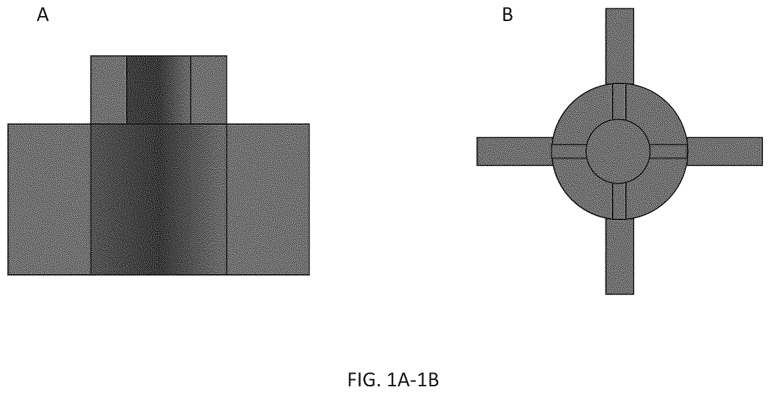

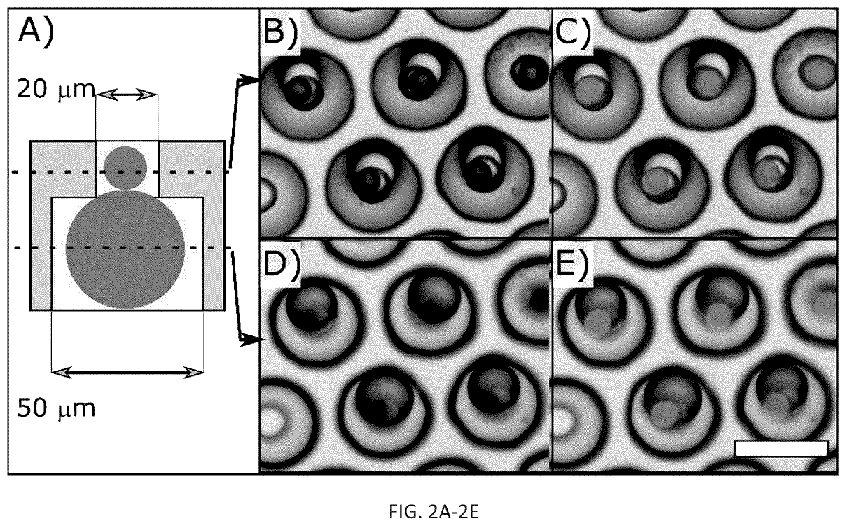

In some embodiments, the microwell array comprises microwells having at least one side wing. Side wings are appendages that extend from the side wall of the microwell. In some embodiments, the side wings permit centrifugation during seal removal of the second surface of the microwell array. Additionally, the side wings allow release of waste/buffer by channeling waste/buffer in from the bottom of the microwell through centrifugation. FIG. 1A shows a cross-section of a microwell comprising side wings. FIG. 1B shows a schematic of a top view of a microwell comprising side wings.

In some embodiments, the microwells on the first surface of the microwell array are different in shape than the microwells on the second surface of the microwell array. In some embodiments, the microwells on the first surface of the microwell array are circular, triangular, rectangular, pentangular, hexangular, heptangular, or octangular in shape while the microwells on the second surface of the microwell array are circular, triangular, rectangular, pentangular, hexangular, heptangular, or octangular in shape.

In some embodiments, the sides of the microwells are non-porous. In some embodiments, the microwell array comprises microwells where the sides of each microwell are hydrophilic. In some embodiments, the microwells of both the first surface and second surface of the microwell array are hydrophobic. In some embodiments, the microwells of both the first surface and second surface of the microwell array are hydrophilic. In some embodiments, the microwells of the first surface of the microwell array are hydrophobic and the microwells of the second surface of the microwell array are hydrophilic. In some embodiments, the microwells of the first surface of the microwell array are hydrophilic and the microwells of the second surface of the microwell array are hydrophobic.

In some embodiments, the sides of the microwells are constructed of the same material as the first and second surface of the array. In some embodiments, the sides of the microwells are constructed of a deposited metal, such as by electrodeposition and/or electroplating of a conductive metal, such as copper and/or nickel.

In some embodiments, the top of the microwell is hydrophobic or fluorophilic.

In some embodiments, the top of the microwell can extend at least partially along an upper surface. The upper surface is the space between the microwell tops of adjacent microwells. In some embodiments, that upper surface is hydrophobic.

In some embodiments, the well bottom of the first surface of the microwell array is porous. In some embodiments, the well bottom of the second surface of the microwell array is porous. In some embodiments, the well bottom of the first surface of the microwell array is porous and the well bottom of the second surface of the microwell array is porous. In some embodiments, the well bottom of the first surface of the microwell is open. In some embodiments, the well bottom of the second surface of the microwell is open. In some embodiments, both the well bottom of the first and second surfaces of the microwell are open.

In some embodiments of the disclosure, well bottom of the first surface comprises pores ranging in size from 0.2 microns up to 10 microns. In some embodiments, the well bottom of the first surface comprises pores in the size of about 0.2 microns. In some embodiments, the well bottom of the first surface comprises pores in the size of about 0.3 microns. In some embodiments, the well bottom of the first surface comprises pores in the size of about 0.4 microns. In some embodiments, the well bottom of the first surface comprises pores in the size of about 0.5 microns. In some embodiments, the well bottom of the first surface comprises pores in the size of about 0.6 microns. In some embodiments, the well bottom of the first surface comprises pores in the size of about 0.7 microns. In some embodiments, the well bottom of the first surface comprises pores in the size of about 0.8 microns. In some embodiments, the well bottom of the first surface comprises pores in the size of about 0.9 microns. In some embodiments, the well bottom of the first surface comprises pores in the size of about 1 micron. In some embodiments, the well bottom of the first surface comprises pores in the size of about 2 microns. In some embodiments, the well bottom of the first surface comprises pores in the size of about 3 microns. In some embodiments, the well bottom of the first surface comprises pores in the size of about 4 microns. In some embodiments, the well bottom of the first surface comprises pores in the size of about 5 microns. In some embodiments, the well bottom of the first surface comprises pores in the size of about 6 microns. In some embodiments, the well bottom of the first surface comprises pores in the size of about 7 microns. In some embodiments, the well bottom of the first surface comprises pores in the size of about 8 microns. In some embodiments, the well bottom of the first surface comprises pores in the size of about 9 microns. In some embodiments, the well bottom of the first surface comprises pores in the size of about 10 microns. In some embodiments, the well bottom of the first surface comprises pores ranging in the size of about 4 microns to about 8 microns. In some embodiments, the well bottom of the first surface comprises pores ranging in the size of about 2 microns to about 2.5 microns. In some embodiments, the well bottom of the first surface comprises pores ranging in the size of about 0.9 microns to about 1.4 microns. In some embodiments, the well bottom of the first surface comprises pores ranging in the size of about 10 microns to about 20 microns.

In some embodiments of the disclosure, the well bottom of the second surface comprises pores ranging in size from 0.2 microns up to 10 microns. In some embodiments, the well bottom of the second surface comprises pores in the size of about 0.2 microns. In some embodiments, the well bottom of the second surface comprises pores in the size of about 0.3 microns. In some embodiments, the well bottom of the second surface comprises pores in the size of about 0.4 microns. In some embodiments, the well bottom of the second surface comprises pores in the size of about 0.5 microns. In some embodiments, the well bottom of the second surface comprises pores in the size of about 0.6 microns. In some embodiments, the well bottom of the second surface comprises pores in the size of about 0.7 microns. In some embodiments, the well bottom of the second surface comprises pores in the size of about 0.8 microns. In some embodiments, the well bottom of the second surface comprises pores in the size of about 0.9 microns. In some embodiments, the well bottom of the second surface comprises pores in the size of about 1 micron. In some embodiments, the well bottom of the second surface comprises pores in the size of about 2 microns. In some embodiments, the well bottom of the second surface comprises pores in the size of about 3 microns. In some embodiments, the well bottom of the second surface comprises pores in the size of about 4 microns. In some embodiments, the well bottom of the second surface comprises pores in the size of about 5 microns. In some embodiments, the well bottom of the second surface comprises pores in the size of about 6 microns. In some embodiments, the well bottom of the second surface comprises pores in the size of about 7 microns. In some embodiments, the well bottom of the second surface comprises pores in the size of about 8 microns. In some embodiments, the well bottom of the second surface comprises pores in the size of about 9 microns. In some embodiments, the well bottom of the second surface comprises pores in the size of about 10 microns. In some embodiments, the well bottom of the second surface comprises pores ranging in the size of about 4 microns to about 8 microns. In some embodiments, the well bottom of the second surface comprises pores ranging in the size of about 2 microns to about 2.5 microns. In some embodiments, the well bottom of the second surface comprises pores ranging in the size of about 0.9 microns to about 1.4 microns. In some embodiments, the well bottom of the second surface comprises pores ranging in the size of about 10 microns to about 20 microns.

In some embodiments, the well bottom of the first surface of the microwell array is porous and the well bottom of the second surface of the microwell array is porous. In some embodiments, the well bottom of the first surface of the microwell array comprises different sized pores than the pores of the well bottom of the second surface of the microarray.

In some embodiments, the microwell array comprises microwells where the top of each microwell is open. An open microwell top means that there is no hinderance between the sidewalls of the microwells at the top of the microwell.

In some embodiments, the microwell array comprises microwells where the bottom of each microwell is open. An open microwell bottom means that there is no hinderance between the sidewalls of the microwells at the bottom of the microwell.

In some embodiments, the microwell array comprises a membrane between the two first surface and second surface in the connection area. In some embodiments, the membrane is porous. In some embodiments, the connection area comprises a porous filter. In some embodiments of the disclosure, the porous filter comprises pores ranging in size from 0.2 microns up to 12 microns. In some embodiments, the porous filter comprises pores in the size of about 0.2 microns. In some embodiments, the porous filter comprises pores in the size of about 0.3 microns. In some embodiments, the porous filter comprises pores in the size of about 0.4 microns. In some embodiments, the porous filter comprises pores in the size of about 0.5 microns. In some embodiments, the porous filter comprises pores in the size of about 0.6 microns. In some embodiments, the porous filter comprises pores in the size of about 0.7 microns. In some embodiments, the porous filter comprises pores in the size of about 0.8 microns. In some embodiments, the porous filter comprises pores in the size of about 0.9 microns. In some embodiments, the porous filter comprises pores in the size of about 1 micron. In some embodiments, the porous filter comprises pores in the size of about 2 microns. In some embodiments, the porous filter comprises pores in the size of about 3 microns. In some embodiments, the porous filter comprises pores in the size of about 4 microns. In some embodiments, the porous filter comprises pores in the size of about 5 microns. In some embodiments, the porous filter comprises pores in the size of about 6 microns. In some embodiments, the porous filter comprises pores in the size of about 7 microns. In some embodiments, the porous filter comprises pores in the size of about 8 microns. In some embodiments, the porous filter comprises pores in the size of about 9 microns. In some embodiments, the porous filter comprises pores in the size of about 10 microns. In some embodiments, the porous filter comprises pores in the size of about 11 microns. In some embodiments, the porous filter comprises pores in the size of about 12 microns. In some embodiments, the porous filter comprises pores ranging in the size of about 4 microns to about 8 microns. In some embodiments, the porous filter comprises pores ranging in the size of about 2 microns to about 2.5 microns. In some embodiments, the porous filter comprises pores ranging in the size of about 0.9 microns to about 1.4 microns. In some embodiments, the porous filter comprises pores ranging in the size of about 0.2 microns to about 12 microns. In some embodiments, the porous filter comprises pores ranging in the size of about 0.2 microns to about 10 microns. In some embodiments, the porous filter comprises pores ranging in the size of about 10 microns to about 20 microns.

The porosity of the porous well bottoms and/or porous filter of the microwell array is used to retain particles such as cells, nuclei, tissue particles and/or pieces to be processed. In some embodiments, the porous well bottoms and/or filter are 7176 sintered glass filter discs porosity D (about 10 microns to about 20 microns), E (about 4 microns to about 8 microns), very fine (about 2 to about 2.5 microns), and/or ultra fine (about 0.9 microns to about 1.4 micron). Alternatively, the porosity of the porous well bottoms and/or porous filter can be of a larger porosity than the entities it needs to retain if it is used in conjunction with beads of the appropriate size that will fill the pores and reduce the effective pore size of the system.

This inception can be used to reduce the overall resistance of the flow through the device while assuring the correct effective pore size. The filter could be made of any material that is compatible with the functional requirements, i.e. hydrophobic bulk and hydrophilic top surface.

In some embodiments, the microwell array is configured to receive vacuum pressure through the bottom of a microwell.

In some embodiments, the microwells of one surface of the array comprise particles, such as genomic barcodes. In some embodiments, the particles are located on beads in the microwells of one surface. In some embodiments, the barcodes are located in the microwells of the first surface of the array.

In some embodiments, the microwell array comprises a sealing device for the first surface of the array. In some embodiments, the sealing device for the first surface is temporary, removable, replaceable, and/or discardable. In some embodiments, the sealing device for the first surface is selected from glass, oil, adhesive, and/or elastomer cover, PCR tape and/or porous membrane.

In some embodiments, the microwell array comprises a sealing device for the second surface of the array. In some embodiments, the sealing device for the second surface is temporary, removable, replaceable, and/or discardable. In some embodiments, the sealing device for the second surface is selected from glass, oil, adhesive, and/or elastomer cover, and/or porous membrane.

In some embodiments, the microwell array comprises a sealing device for the first and second surface of the array. In some embodiments, the sealing device for the first surface of the array is the same as the sealing device for the second surface of the array. In some embodiments, the sealing device for the first surface is temporary, removable, replaceable, and/or discardable. In some embodiments, the sealing device for the first surface is constructed from glass, oil, adhesive, and/or elastomer cover, and/or porous membrane. In some embodiments, the sealing device for the second surface is temporary, removable, replaceable, and/or discardable. In some embodiments, the sealing device for the second surface is constructed from glass, oil, adhesive, and/or elastomer cover, and/or porous membrane. In some embodiments, the sealing device for the first surface is temporary, removable, replaceable, and/or discardable while the sealing device for the second surface is temporary, removable, replaceable, and/or discardable. In some embodiments, the sealing device for the first surface is constructed from glass, oil, adhesive, and/or elastomer, and/or porous membrane cover while the sealing device for the second surface is constructed from glass, oil, adhesive, and/or elastomer cover, and/or porous membrane.

### Method of Barcoding

In another aspect, the present disclosure is directed to a method of barcoding the genetic material of particles, the method comprising three steps: 1) loading one particle to one of a plurality of wells of a microwell array where the microwell array comprises a first surface, a second surface, and a connection area, where the first surface and second surface meet at the connection area; each surface of the microwell array comprises a plurality of independent wells wherein: each well comprises a top, bottom, and sides; each well of the first surface is connected to a corresponding well of the second surface, wherein the wells are configured so that the bottom of the well of the first surface is in contact with the bottom of the well of the second surface and the contact takes place at the connection area; and wherein the microarray enables a the coupling of one particle with at least one bead; 2) processing the barcode to particle coupling; 3) collecting barcoded genetic material. In one embodiment, the method further comprises the steps of: a) loading particles in the wells of the first surface of the microwell array; b) sealing the first surface of the microwell array; c) flipping over the microwell array so that the second surface is accessible; wherein steps a, b, and c occur in succession before step 1. In some embodiments, step a) further comprises loading the particles into the wells of the first surface of the microwell array by loading a singular capture bead from a plurality of capture beads into a well of the microwell array, wherein the capture beads comprise the particles. In some embodiments, the plurality of capture beads is selected from hydrogel, alginate, silica, glass, magnetic, polystyrene, polyactide, and polyacrylamide. In some embodiments, step a) is performed with a vacuum applied to the microarray. In some embodiments, the method further comprises removal of excess loaded particles; wherein the removal of excess loaded particles occurs after step a but before step b.

In another aspect, the present disclosure is directed to a flip array method of barcoding the genetic material of particles, the method comprising three steps: 1) loading of at least one particle to one of a plurality of wells of a microwell array where the microwell array comprises a first surface, a second surface, and a connection area, where the first surface and second surface meet at the connection area; each surface of the microwell array comprises a plurality of independent wells wherein: each well comprises a top, bottom, and sides; each well of the first surface is connected to a corresponding well of the second surface, wherein the wells are configured so that the bottom of the well of the first surface is in contact with the bottom of the well of the second surface and the contact takes place at the connection area; and wherein the microarray enables a the coupling of one particle with at least one bead; 2) processing the barcode to particle coupling; 3) collecting barcoded genetic material. In one embodiment, the method further comprises the steps of: a) vacuum or flow-driven loading particles, such as genomic barcodes in the wells of the first surface of the microwell array; b) sealing the first surface of the microwell array with a porous or non-porous membrane; c) flipping over the microwell array so that the second surface is accessible; d) loading particles in empty microwells using gravity only with non-porous membranes or using gravity or vacuum-driven loading with a porous membrane; e) optionally sealing the microwell to proceed with cell lysis, molecule capture by the beads and unsealed to perform wash and buffer exchange steps to lead to biomolecular and biochemical reactions; wherein steps a, b, c, d and e occur in succession before step 1.

In some embodiments, the processing step comprises: lysing cells; capturing mRNA molecules through a heating and cooling cycle; releasing the genetic barcodes; hybridizing the genomic material of the cell to the genetic barcode. In some embodiments, the processing step comprises: lysing cells; capturing mRNA molecules through a heating and cooling cycle; hybridizing the genomic material of the cell to the genetic barcode; and releasing the genetic barcodes. In some embodiments, the method further comprises exposing the microarray to a form of centrifugation. In some embodiments, the method further comprises exposing the microarray to a vacuum. In some embodiments, the centrifugation occurs at 100-10,000×g. In some embodiments, the centrifugation occurs at 100-1,000×g. In some embodiments, the centrifugation occurs at 100×g. In some embodiments, the centrifugation occurs at 100× g. In some embodiments, the centrifugation occurs at 100× g. In some embodiments, the centrifugation occurs at 100× g. In some embodiments, the centrifugation occurs at 100× g. In some embodiments, the centrifugation occurs at 100× g. In some embodiments, the centrifugation occurs at 100× g. In some embodiments, the centrifugation occurs at 100× g. In some embodiments, the centrifugation occurs at 100× g. In some embodiments, the centrifugation occurs at 1,000×g. In some embodiments, the centrifugation occurs at 1,100×g. In some embodiments, the centrifugation occurs at 1,200×g. In some embodiments, the centrifugation occurs at 1,300×g. In some embodiments, the centrifugation occurs at 1,400×g. In some embodiments, the centrifugation occurs at 1,500×g. In some embodiments, the centrifugation occurs at 2,000×g. In some embodiments, the centrifugation occurs at 2,500×g. In some embodiments, the centrifugation occurs at 3,000×g. In some embodiments, the centrifugation occurs at 3,500×g. In some embodiments, the centrifugation occurs at 4,000×g. In some embodiments, the centrifugation occurs at 4,500×g. In some embodiments, the centrifugation occurs at 5,000×g. In some embodiments, the centrifugation occurs at 5,500×g. In some embodiments, the centrifugation occurs at 6,000×g. In some embodiments, the centrifugation occurs at 6,500×g. In some embodiments, the centrifugation occurs at 7,000×g. In some embodiments, the centrifugation occurs at 7,500×g. In some embodiments, the centrifugation occurs at 8,000×g. In some embodiments, the centrifugation occurs at 8,500×g. In some embodiments, the centrifugation occurs at 9,000×g. In some embodiments, the centrifugation occurs at 9,500×g. In some embodiments, the centrifugation occurs at 10,000×g.

In some embodiments, the method further comprises step 1a) sealing the loaded cells in the wells of the second surface of the microwell array; wherein step 1a occurs after step 1 and before step 2.

In some embodiments, the method further comprises a step of adding one or more buffers and/or reagents to at least one well; where the at least one well comprises a particle such as at least one genetic barcode; and wherein the step of adding one or more buffers and/or reagents to the at least one well occurs after step 1 and before step 2. In some embodiments, the one or more buffers and/or reagents comprise a lysing agent.

In some embodiments, the method further comprises a step of step of adding one or more buffers and/or reagents to at least one well; where the at least one well comprises a particle such as at least one genetic barcode; and wherein the step of adding one or more buffers and/or reagents to the at least one well occurs step 1a. In some embodiments, the one or more buffers and/or reagents comprise a lysing agent. In some embodiments, the method further comprises opening the array and washing the barcoded genomic material. In some embodiments, the method further comprises, a step of removing excess cells from the second surface of the microarray, wherein the step of removing excess cells occurs after step 1 and before step 2.

In some embodiments, the method further comprises performing reverse-transcription (RT) with reverse template switch. In some embodiments, the method further comprises performing polymerase chain reaction directly on the array or after collecting barcoded genomic material. In some embodiments, the method further comprises the sequencing of collected genomic material.

In some embodiments, the disclosed microwell array includes a frit filter made of a glass (or other suitable material). Crosstalk through the filter base to other microwells is reduced or prevented by control of surface properties.

In some embodiments, the microwell arrays of this disclosure can be used to array single-cells. Microwells can be isolated from each other and thus used to easily create a large number (100,000's) of independent assays.

It is noted that the first surface and second surface of the microwell array described herein may have different properties while being constructed of the same material. The properties of the first and second surfaces of the microwell array are a result of the sequential etching process further described herein. For example, the sidewall of the wells of one surface may be hydrophilic and non-porous while the bottom of the wells is hydrophobic and porous. In some embodiments, the sidewall of the wells of one surface may be hydrophilic and non-porous while the bottom of the wells is hydrophobic and porous. In some embodiments, the sidewall of the wells of one surface may be hydrophilic and non-porous while the bottom of the wells is hydrophilic and porous. In some embodiments, the sidewall of the wells of one surface may be hydrophilic and porous while the bottom of the wells hydrophilic and porous.

The methods and model of the present disclosure will be better understood by reference to the following Examples, which are provided as exemplary of the disclosure and not in any way limiting.

### EXAMPLES

### Example 1: Microwell Fabrication and Proof of Concept

A microwell array of 26,535 microwells was constructed with 50 μm/20 μm bead/cell microwell dimensions. A 5:1 PDMS (Sylgard, Dow Corning) mold was used to pattern a UV and heat-curable epoxy adhesive (Norland Optics) layered on top of a PDMS coated glass slide. After crosslinking, a precut piece of PCR tape was affixed to give it mechanical strength and allow robust manipulation. The device was placed onto a glass frit connected to a controlled vacuum for drying purpose. 30 μm polylactide beads (Corpuscular) were loaded onto the Array. After bead sedimentation, the array was dried by vacuum and the bead excess removed with Magic Tape (Scotch). Then, a piece of PCR tape was applied to lock the capture beads. The array was flipped and the wells of the second surface of the array were loaded with 15 μm red fluorescent polystyrene microspheres (Life Technologies) to mimic mammalian cells. Every step was monitored by macroscope (AZ-100M, Nikon), which allows high-resolution imaging of a large field of view.

The design was validated through the data showing perfect cell-bead pairing (FIG. 2). Several improvements were identified to the loading statistics, including loading barcoded beads under aspiration and control the wetting at the device-PCR tape interface. Optimization was then performed to the microwell dimensions.

The microwell array was fabricated using UC curable materials, such as Norland Optics Adhesive (NOA) or Norland Electronic Adhesives (NEA). A mold made of the silicon elastomer PolyDiMethylSiloxane (PDMS) was obtained by soft-lithography (Duffy. D. C. Duffy, J. C. McDonald, O. J. A. Schueller, G. M. Whitesides, Anal. Chem., 1998, 70, 4974-4984).)

After placing some NEA 121 Red on a PDMS coated slide, we applied the mold and added some mechanical pressure. Mechanical pressure is supplied that avoids buckling of the die as appreciated by the skilled artisan. The assembly was then exposed to UV illumination for 8 minutes. The device can be easily demolded and manipulated. The mold can alternatively be made with perfluoropolymers, such as the Fluorolink® MD 700 (Solvay) or FluorAcryl 2000MA (Cytonix).

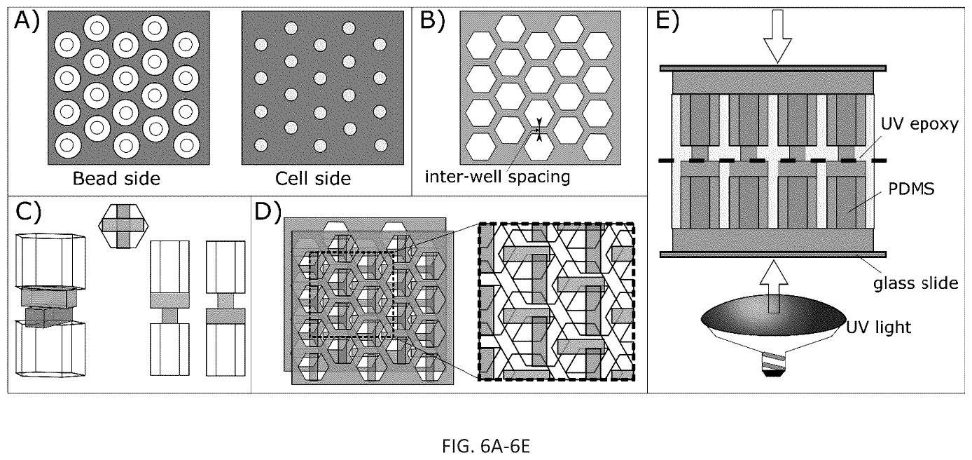

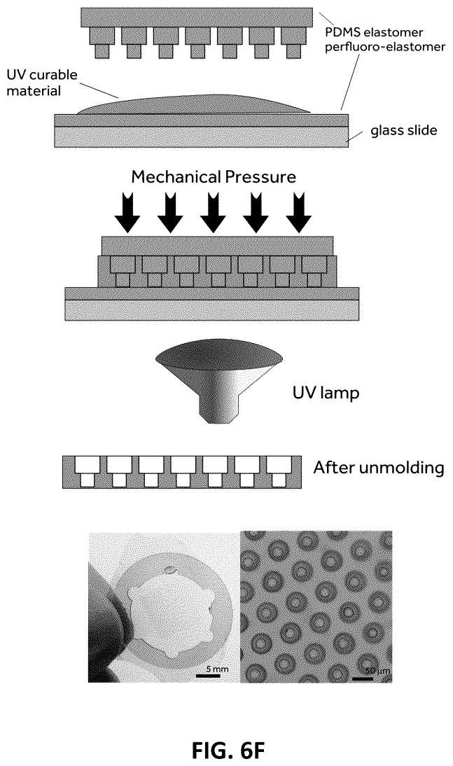

Fabrication of the microwell was achieved as follows: A) NEA 121Red was dispensed on a PDMS-coated slide, and a PDMS die was applied to the PDMS coated glass slide. B) The sandwich was pressed so that no NEA 121Red is present between the tip and the PDMS coated glass side to ensure through holes. The assembly was then exposed to UV light for 8 minutes to crosslink the NEA 121Red. C) The membrane/device can be easily unmolded. D) Example of a device created using the protocol. The design includes a larger ring around the array to add mechanical stability. The insert shows a zoomed-in image of the array where the through holes are distinguishable (FIG. 6F).

### Example 2: Microwell Array Performance for Single-Cell Transcriptomics

Single-cell transcriptomics will be developed and validated. The hypotheses that 1) small reactor volumes drive a higher capture rate of mRNA molecules and cDNA yield, 2) shear-free methods reduce crosscontamination will be tested. Various barcoded beads such as polystyrene beads (ChemGenes), hydrogel beads, and dissolvable beads will be tested to test if cDNA conversion is more efficient in solution. Experiments will be conducted with a focus on quality of sequencing data per single cell and benchmark the disclosed platform against established (Bag-seq), Seq-Well and commercial Chromium (10× Genomics) methods.

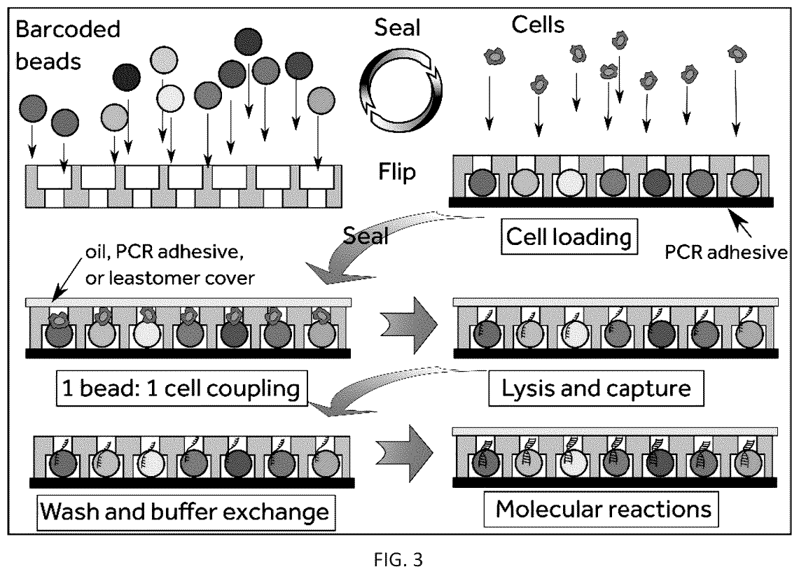

The workflow of FIG. 3 will be developed where: 1) barcoded capture beads are loaded; 2) the bead array is sealed; 3) the array is flipped; 4) cells are loaded; 5) the second surface of the array is sealed; 6) cells are lysed and mRNA captured by a heating (50° C.) and a slow cooling cycle; 7) the second surface of the array is opened and washed; 8) reverse-transcription (RT) with reverse template switch is performed; 9) PCR is performed directly on the array or after collecting beads.

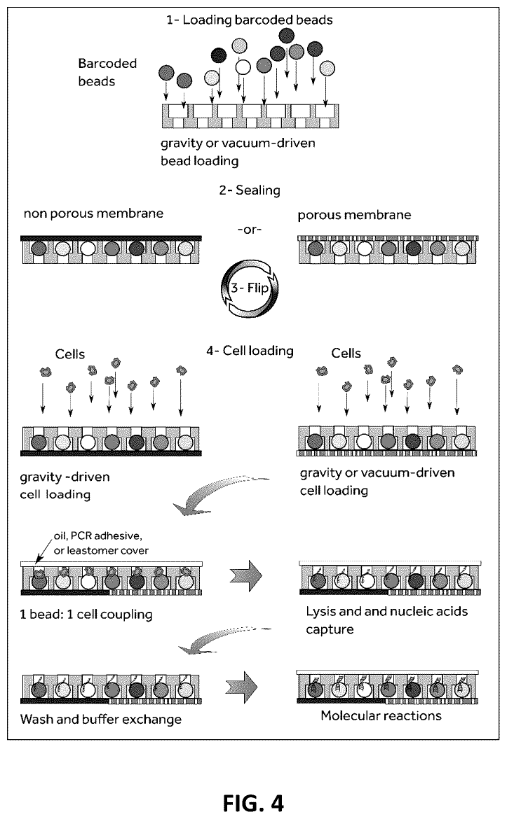

In another workflow of FIG. 4 a) vacuum or flow-driven particles, i.e. genomic barcodes are loaded in the wells of the first surface of the microwell array; b) the first surface of the microwell array is sealed with a porous or non-porous membrane; c) the microwell array is flipped over so that the second surface is accessible; d) particles are loaded in empty microwells using gravity only with non-porous membranes (FIG. 4, left panel) or using gravity or vacuum-driven loading with a porous membrane (FIG. 4, right panel); e) and the microwells are optionally to proceed with cell lysis, molecule capture by the beads and unsealed to perform wash and buffer exchange steps to lead to biomolecular and biochemical reactions (FIG. 4).

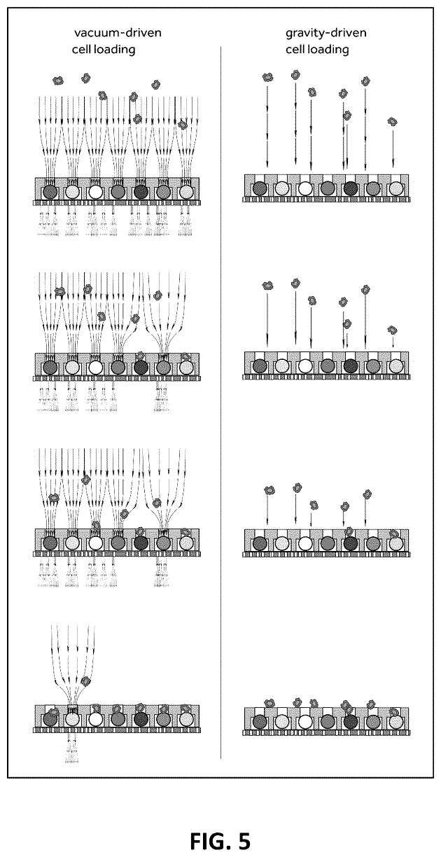

In one embodiment, particles are cells which are actively loaded with a vacuum. With vacuum-driven loading, cells follow the flow streamlines. If a microwell is open (without cell), streamlines go through the microwell. If a microwell contains a cell, streamlines do not go through the microwell and cells are diverted to the next empty microwell. In gravity-driven loading, cells sediment following a straight downward trajectory. Another difference in those cell loading modes reside in the fact that the microwell size ensures single-cell loading in gravity-driven scheme, while the constraint is relieved in the vacuum-driven scheme. As a result, the vacuum driven scheme can accommodate a wider range of cell sizes (FIG. 5)

Hydrogel beads will be polyacrylamide bags, or polyacrylamide beads crosslinked with cleavable disulfide bridges, manufactured with a simple droplet generator to assure their monodispersity. An acrydite modified ssDNA is incorporated to serve as the initial template for split-pooling barcoding. Solid beads will be amine coated polystyrene beads. The solid beads will be covalently coated with a brush of poly-L-lysine using the crosslinker bis(sulfosuccinimidyl) suberate (BS3). Following coating, the initial amino-terminated ssDNA are coated using BS3 on the poly-L-lysine. Alternatively, solid barcoded capture beads will be used. Split pooling will add a universal primer 1 (UP1), a unique molecular identifier (UMI or varietal tag), a first barcode byte, and a common adapter sequence. Every round will add a barcode byte, while the last round will append an additional poly-dT capture probe.

The RT reaction adds the same sequence of bases to all cDNAs due to the terminal deoxynucleotidyl transferase activity of the enzyme. This allows the addition of a universal primer 2 (UP2) to the cDNAs and their subsequent PCR amplification. The amplicons are then fragmented using the Nextera XT kit. In paired-end sequencing, Read 1 will correspond to the 3′ end of the captured nucleic acid and be used for mapping the genome. Read 2 will correspond to the 5′ end of the captured nucleic acid, the varietal tag, and the barcode used to collapse sequencing reads by their common single-cell origin.

Devices will be manufactured in optical adhesive as described in Example 1. The well dimensions will be optimized for both the barcoded capture beads and cells. The wells surface properties will be adjusted with PEG-silane reagents to prevent the adsorption of nucleic acids. Barcoded capture beads will be loaded under aspiration. Under aspiration conditions, loading is active and driven by the flow through empty microwells. This method optimizes loading, minimizes bead waste, and optimizes footprint as it alleviates the design criterion of 1.5 to 5-fold the size of the particle for the microwell diameter.

The transcriptomes of mammalian cells will be characterized through the disclosed methods. The mammalian cells used for transcriptome characterization include normal skin fibroblast cells (Malme-3) and the tumor counterparts (Malme-3M), three breast tumor cell lines (SK-BR-3, BT-20, MCF-7) used to characterize the Bag-seq method, the human kidney lymphocyte cell line HEK293T, used as a reference to compare single-cell genomic methods. The mouse fibroblast cell line NIH3T3 will be used for mixed-species experiments.

Cross-contamination occurrence in the disclosed platform will be tested. Cross-contamination relies on perfect sealing on both surfaces of the array to avoid cross contamination. To evaluate cross-contamination, a one-step RT-PCR will be performed on the highly expressed actin gene using sparsely loaded pre-stained single-cells. An intercalating dye will be used to report the fluorescence around cell-laden microwells. Hence, a fluorescent signal in cell-free microwells would indicate either contamination from 1) cell-free nucleic acids or 2) crosstalk from adjacent cell-occupied microwells, depending on their proximity to cell-laden microwells. Additionally, a mixture of human and mouse cells will be analyzed to further test cross-contamination. These experiments will permit quantification of cross-contamination and test the advantage of disclosed shear-free methods.

Data will show the number of uniquely tagged templates, identified genes, and the number of reads per gene after clustering varietal tags. Lastly, a principal component analysis will be performed to test the consistency of the gene expression detected between single cells of the same cell line. In parallel, generated bulk RNA-seq profiles of the cell lines will serve as references. Finally, the disclosed method will be benchmarked against common Bag-seq, Seq-Well and the commercial approaches, i.e. 10× Genomics Chromium.

Two possible pitfalls relate to the surface properties of the array 1) sealing quality and 2) potential adsorption of nucleic acids onto the microwell walls. To address these potential problems, UV curable epoxy (NOA) will be used. The surface properties of the UV curable epoxy can be tailored with silane reagents after ozone oxidation. Differential surface treatment between the microwell inner walls and the array faces will be performed through stamping. Alternatively, polycarbonate can be used to create the disclosed array. Both NOA and polycarbonate are hydrophilic and will help microwell filling.

Experiments should show an absence of microwell crosstalk. Additionally, results are expected to show detection of at least 5,000 uniquely expressed genes in HEK293T in par with the 10× Genomics Chromium system.

### Example 3: Optimizing Cell Capture Rate

The study of rare cell populations requires high-throughput and high cell capture rates. Cells can sediment and get captured in microwells only if they land within their surface area. The cell capture rate depends directly on the surface fraction of the microwells, and it decreases with an increase of surface between the microwells.

Microwell arrays allowing for smaller barcoded beads will be designed. These designs will lead to an increased surface density of capture microwells and will increase the cell capture rate. Cell capture rate will be tested in a microwell array of hexagonal microwells. The cell capture rate will be measured as a function of the inter-well distance (FIG. 6B) and seeding concentration using 15 μm fluorescent microbeads and HEK293T fluorescently labeled with Alexa488-wheat germ agglutinin. A measurement will be obtained for the number of beads/cells inside and between microwells, including before and after washing. This will aide in reporting the single cell capture as a function of array occupancy. An increase in capture rate is expected when inter-well distance is decreased and seeding concentration increased. This experiment will establish the limit of the single cell capture rate and quantify the impact of deviating from the best-case scenario. The single cell capture rate of the disclosed microwell arrays will be measured and compared to the single cell capture rate of current methods (20% to 80% for droplet technologies and close to 100% for microwell methods but at only 10% array occupancy).

Hexagon-shaped microwells terminated by a trench at their bottom will be used to increase the microwell density of the microwell array. The cell and bead microwells will have the same design, but the trench will be rotated 90°, as shown in FIG. 6C. This design will assure connection and 1:1 correspondence between the two microwell types even if the surfaces of the array are grossly misaligned (FIG. 6D). The design will be readily manufacturable using our current method (FIG. 6E).

A consequence of using smaller microwells is the subsequent requirement to use smaller barcoded beads. Smaller barcoded beads can be challenging to synthesize due to the viscosity of the hydrogel solution. However, an advantage of the disclosed microarray is that the disclosed array enables for barcoded beads to be loaded under aspiration. As a result, the deformable hydrogel beads can be squeezed into microwells with smaller diameters. The design relies on the robust and array-wide creation of a conduit between the microwell types which may prove challenging. If necessary, either plasma etching or dry reactive ion-etching will be used to remove any thin layer of material blocking the conduits. The success rate of the conduit manufacturing will be determined by aspirating a fluorescent solution and report if it fills the entire connected microwells.

An objective of the disclosed microwell array optimization is to reliably pair 10,000 single-cells with barcoded beads with a cell capture rate of 90% and a barcoded bead usage greater than 50%. Such data would be an order of magnitude greater than typical bead based barcoding methods. Increasing the throughput on the platform will be simplified by increasing its overall area.

### Example 4: Microwell Array for Single Nuclei Applications

Creating a single cell solution from multicell solid tissues poses several challenges: 1) it can bias the analysis towards cells that can sustain the procedure, 2) it is prone to generate cross-contamination due to materials released in solution from sheared cells, 3) it is impossible to perform for certain tissues such as brain tissues due to the neuron morphology. In contrast to single cells, single nuclei can be easily collected from most tissues, including embedded clinical samples. Nuclei are a source of both genomic DNA and mRNA. Genomic DNA can be used to generate copy number variant (CNV) profiles to reveal genomic instabilities and infer tumor evolution. Nuclear mRNA is sufficient to establish cell type and state. The disclosed microarray will be optimized for concurrent genomic and transcriptomic analysis of single nuclei. The disclosed method will be validated on cell lines and clinical samples from prostate cancer patients will be characterized according to the disclosure.

Method: mammalian cell nuclei are about 6 μm in diameter, which provides a target size of 9 to 12 μm diameter and depth for the microwell dimensions. The malleability property of the hydrogel beads will be used to squeeze smaller hydrogel beads into smaller diameter microwells. This will overcome the design constraints on the barcoded beads while loading them under aspiration. A dilute solution of barcoded beads will continuously be loaded. The continuously loaded dilute solution of barcoded beads will be used to optimize loading.

The nuclei capture rate will allow evaluation of the utility of the disclosed microwell array. Nuclei capture rate will be measured through the use of a solution of nuclei obtained by the freeze-thaw method and pre-stained with a DNA fluorescent dye. Next, CNV profiles will be generated from single nuclei solution prepared from a mixture of cell lines. Genomic DNA will be captured using random T/G primers and extended with DNA polymerase. Coverage statistics will be measured, including the number of mappable paired-end sequencing reads, the amount of genome covered by the nuclei, and the number of reads and the genome coverage per single nucleus.

A protocol will be developed in order to perform concurrent DNA and mRNA analysis on single nuclei. DNA and mRNA will be captured in mild denaturation conditions, and the extension will be performed in conditions where both DNA and cDNA synthesis can occur. Sequencing reads will be attributed to either DNA or mRNA based on the genomic regions (exon, intron, intergenic) from which they map.

Additional libraries will be generated based on DNA and mRNA. These libraries will be used as references to validate the mapping method. The data obtained from the multi-omic approach will be compared to that of individual CNV and transcriptomic methods. Finally, analyzation of clinical samples from healthy donors and patients with prostate cancer will occur.

Resolution and aspect ratio of the array to be microfabricated for single nuclei applications are important.

It is expected that the disclosed microwell array and methods will generate CNV profiles with performance similar to the Bag-seq method, including 500 k unique reads for a genome coverage of 1.6% per single nucleus. Additionally, the disclosed microwell array and methods are expected to generate CNV profiles on at least 10,000 single nuclei on a single microwell array. Lastly, it is expected that a single microwell array of the disclosure will allow CNV and transcriptomic profiles of at least 10,000 individual nuclei.

The described embodiments and examples of the present disclosure are intended to be illustrative rather than restrictive and are not intended to represent every embodiment or example of the present disclosure. While the fundamental novel features of the disclosure as applied to various specific embodiments thereof have been shown, described and pointed out, it will also be understood that various omissions, substitutions and changes in the form and details of the devices illustrated and in their operation, may be made by those skilled in the art without departing from the spirit of the disclosure. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or embodiment of the disclosure may be incorporated in any other disclosed or described or suggested form or embodiment as a general matter of design choice. Further, various modifications and variations can be made without departing from the spirit or scope of the disclosure as set forth in the following claims both literally and in equivalents recognized in law.

1. A microwell array comprising:
a first surface, a second surface, and a connection area, where the first surface and second surface meet at the connection area;
each surface of the microwell array comprise a plurality of independent wells wherein:
each well comprises a top, bottom, and sides;
each well of the first surface is connected to a corresponding well of the second surface, wherein the wells are configured so that the bottom of the well of the first surface is in contact with the bottom of the well of the second surface and the contact takes place at the connection area; and wherein the microarray enables a coupling of one particle with at least one additional particle.

2. The microwell array of claim 1, wherein the microwells are circular, triangular, rectangular, pentangular, hexangular, heptangular, or octangular in shape.

3. The microwell array of claim 1, wherein the top of each well is open.

4. The microwell array of claim 1, wherein the bottom of each well is porous.

5. The microwell array of claim 1, wherein the well sides are non-porous.

6. The microwell array of claim 1, wherein the wells have a diameter of at least about 1.25 to 5-fold the size of a particle to be placed in the well.

7. The microwell array of claim 6, wherein the wells have a diameter of at least about 1.5-fold the size of a particle to be placed in the well.

8. The microwell array of claim 1, wherein the wells have a diameter of at least 5 μm.

9. The microwell array of claim 1, wherein the diameter of the wells on the first surface and the diameter of the wells on the second surface is a different diameter.

10. The microwell array of claim 1, wherein the diameter of the wells on the first surface and the diameter of wells on the second surface is the same diameter.

11. The microwell array of claim 1, wherein the wells have a depth that is smaller than the size of a particle to be placed in the well.

12. The microwell array of claim 1, wherein the wells have a depth that is larger than the size of a particle to be placed in the well.

13. The microwell array of claim 1, wherein the wells have a depth within 0.9 to 5-fold the size of a particle to be placed in the well.

14. The microwell array of claim 13, wherein the wells have a depth of about 1.5-fold the size of a particle to be placed in the well.

15. The microwell array of claim 1, wherein the depth of the wells on the first surface and the depth of the wells on the second surface is a different depth.

16. The microwell array of claim 1, wherein the depth of the wells on the first surface and the depth of the wells on the second surface is the same depth.

17. The microwell array of claim 4, wherein the porous well bottom of the first surface comprises pores ranging in size from 0.2 microns to 20 microns.

18. The microwell array of claim 17, wherein the porous well bottom of the first surface comprises pores ranging in size from 0.2 microns to 10 microns.

19. The microwell array of claim 17, wherein the porous well bottom of the first surface comprises pores ranging in size from 0.2 microns to 5 microns.

20. The microwell array of claim 4, wherein the porous well bottom of the second surface comprises pores ranging in size from 0.2 microns to 20 microns.

21. The microwell array of claim 20, wherein the porous well bottom of the second surface comprises pores ranging in size from 0.2 microns to 10 microns.

22. The microwell array of claim 21, wherein the porous well bottom of the second surface comprises pores ranging in size from 0.2 microns to 5 microns.

23. The microwell array of claim 1, wherein the wells of the first surface comprise particles.

24. The microwell array of claim 23, wherein the particles are located on beads in the wells of the first surface.

25. The microwell array of claim 1, further comprising a sealing device for the first surface.

26. The microwell array of claim 25, wherein the sealing device for the first surface is temporary, removable, replaceable, and/or discardable.

27. The microwell array of claim 25, wherein the sealing device for the first surface is selected from glass, oil, adhesive, elastomer cover, and porous membrane.

28. The microwell array of claim 1, further comprising a sealing device for the second surface.

29. The microwell array of claim 28, wherein the sealing device for the second surface is temporary, removable, replaceable, and/or discardable.

30. The microwell array of claim 28, wherein the sealing device for the second surface is selected from glass, oil, adhesive, elastomer cover, and porous membrane.

31. A method of barcoding the genetic material of single cells, the method comprising:
1) loading one cell to one of a plurality of wells of a microwell array of claim 1;
2) processing the barcode to cell coupling;
3) collecting barcoded genetic material.

32. The method of claim 31, further comprising the steps of:
a) loading particles in the wells of the first surface of the microwell array;
b) sealing the first surface of the microwell array;
c) flipping over the microwell array so that the second surface is accessible;
wherein steps a, b, and c occur in succession before step 1.

33. The method of claim 32, wherein step a) further comprises loading the particles into the wells of the first surface of the microwell array by loading a singular capture bead from a plurality of capture beads into a well of the microwell array, wherein the capture beads comprise the particles.

34. The method of claim 33, wherein the plurality of capture beads are selected from hydrogel, silica, glass, magnetic, alginate, polystyrene, polylactide, and polyacrylamide.

35. The method of claim 32, further wherein step a) is performed with a vacuum applied to the microarray.

36. The method of claim 32, further comprising removal of excess loaded particles; wherein the removal of excess loaded particles occurs after step a but before step b.

37. The method of claim 31, wherein the processing step comprises:
lysing cells;
capturing mRNA molecules through a heating and cooling cycle;
releasing the genetic barcodes;
hybridizing the genomic material of the cell to the genetic barcode.

38. The method of claim 31, wherein the processing step comprises:
lysing cells;
capturing mRNA molecules through a heating and cooling cycle;
hybridizing the genomic material of the cell to the genetic barcode;
releasing the genetic barcodes.

39. The method of claim 31, further comprising exposing the microarray to a form of centrifugation.

40. The method of claim 31, further comprising:
1a) sealing the loaded cells in the wells of the second surface of the microwell array; wherein step 1a occurs after step 1 and before step 2.

41. The method of claim 31, further comprising a step of adding one or more buffers and/or reagents to the well; and
wherein the step of adding one or more buffers and/or reagents to the well occurs after step 1 and before step 2.

42. The method of claim 33, further comprising a step of adding one or more buffers and/or reagents to the well; and
wherein the step of adding one or more buffers and/or reagents to the well occurs before step 1a.

43. The method of claim 41, wherein the one or more buffers and/or reagents comprise a lysing agent.

44. The method of claim 31, further comprising opening the array and washing the barcoded genomic material.

45. The method of claim 31, further comprising a step of removing excess cells from the second surface of the microarray, wherein the step of removing excess cells occurs after step 1 and before step 2.

46. The method of claim 31, further comprising performing reverse-transcription (RT) with reverse template switch.

47. The method of claim 31, further comprising performing polymerase chain reaction directly on the array or after collecting barcoded genomic material.

48. The method of claim 31, further comprising sequencing of captured genomic material.

49. The microwell array of claim 1, wherein the particles comprise genomic barcodes.

50. The microwell array of claim 1, wherein the particles comprise beads.

51. The microwell array of claim 1, wherein the particles comprise cells.
